(19) United States
(12) Patent Application Publication
(10) Pub. No.: US 2020/0323838 A1
(43) Pub. Date: Oct. 15, 2020

(54) LENALIDOMIDE IMMEDIATE RELEASE FORMULATIONS

(71) Applicant: Synbias Pharma AG, Schaffhausen (CH)

(72) Inventors: Oleksandr Zabudkin, Gernsbach (DE); Vladimir Matha, Bor?ov nad Vltavou (CZ); Iaroslav Matviienko, Mannheim (DE)

(21) Appl. No.: 16/758,044

(22) PCT Filed: Oct. 26, 2018
(86) PCT No.: PCT/EP2018/079480

§ 371 Date: Apr. 22, 2020

Related U.S. Application Data
(60) Provisional application No. 62/577,302, filed on Oct. 26, 2017.

(51) Int. Cl. A61K 31/454; A61K 9/00; A61K 47/26; A61K 47/38; A61K 47/36; A61K 47/12

(52) U.S. Cl. CPC A61K 31/454; A61K 9/0053; A61K 47/12; A61K 47/38; A61K 47/36; A61K 47/26

(57) ABSTRACT

The present invention provides for a pharmaceutical composition comprising lenalidomide form A, a filler and a disintegrant and a superdisintegrant. The present invention further relates to a process for the preparation of said composition. The composition according to the invention may be used as medicament, particularly for the treatment of multiple myeloma and myeolodisplastic syndromes.

Lenalidomide, [INN] is 3-(4-amino-1,3-dihydro-1-oxo-2H-isoindol-2-yl)-2,6-piperidine dione, FIG. 1, is known to inhibit the proliferation of certain haematopoietic tumour cells, promote the immunity mediated by T-cells and natural-killer (NK) cells, stimulate erythropoiesis, inhibit angiogenesis and the production of pro-inflammatory cytokines such as TNF-α and interleukin-6 and 12 (Kotla V, Goel S, Nischal S, et al. Mechanism of action of lenalidomide in hematological malignancies. Journal of Hematology & Oncology. 2009; 2:36. doi:10.1186/1756-8722-2-36, for review).

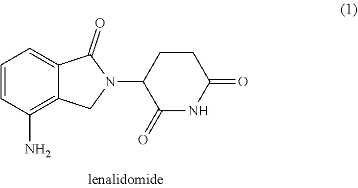 [SMILES: Nc1cccc2c1CN(C1CCC(=O)NC1=O)C2=O]

Lenalidomide is soluble in organic solvent/water mixtures, and buffered aqueous solvents and exhibits the highest solubility of 18.0 mg/ml in 0.1 HCl buffer. Solubility in less acidic buffer is decreasing to about 0.4 to 0.5 mg/ml.

Besides pH, lenalidomide solubility is influenced by molecule polymorphisms. As evident from U.S. Pat. No. 7,465,800, polymorph A was characterized as the most soluble one with a solubility of 6.2 mg/ml in a medium of HCl buffer pH 1.8, containing 1% sodium lauryl sulfate. Polymorph B and E revealed solubility of 5.8 mg/ml and 4.7 mg/ml, respectively, under the same experimental conditions.

Lenalidomide is characterized as class III drug (high solubility, low permeability) based on BCS (FDA, 2005, Clinical Pharmacology and Biopharmaceutics reviews, Appl. Number 21-880, page 3). In this class of compounds, the absorption rate limiting step is the permeability of the drug substance rather than the rate of dissolution of the drug formulation.

Currently, lenalidomide is approved for the treatment of multiple myeloma and myelodysplastic syndromes. It is marketed under the name REVLIMID® by Celgene in the form of hard gelatin capsules since 2004.

Beside the use in hematooncology, lenalidomide showed also promising results in experimental treatment of Alzheimer disease in mice (Alzheimer's & Dementia, ISSN: 1552-5260, Vol: 9, Issue: 4,2013).

The compound and its process were disclosed in U.S. Pat. No. 5,635,517. The following patents are solving synthesis, crystallinity and final dosage forms of lenalidomide.

Lenalidomide can exist in different polymorphic forms, which may differ from each other in terms of stability, physical properties, spectral data and methods of preparation. WO 2005023192 disclosed crystalline Form A, Form B, Form C, Form D, Form E, Form F, Form G and Form H of lenalidomide. Each polymorphic form is characterized by individual XRD pattern. For example, Form A has significant peaks at 8, 14.5, 16, 17.5, 20.5, 24 and 26 degrees 20. Form B is dearly distinguished from form B and has significant peaks at 16, 18, 22 and 27 degrees 2Θ.

These forms can interconvert, e.g. hemihydrate B converts into dihydrate E in the presence of water, anhydrate A converts into form E in the presence of an amount of form E or in the presence of water.

The original formulation is declared to use micronized polymorph B, to ensure the necessary content uniformity, neither the particle size (when included in the distribution range of micronized particles), neither the polymorphic form affect the dissolution of the capsules (EMEA “Scientific Discussion” for Revlimid, 2007). The micronization of API is related with several disadvantages like e.g. high energy input, potential increase of toxicity, health and safety risk during preparation and handling, sensitivity to moisture and oxidation due to enlarged surface area, and flowability problems due to agglomeration tendency of micronized drug. WO2010054833 discloses a solid solution of lenalidomide in a matrix material. These solid solutions are prepared using melt extrusion and spray drying. These pharmaceutical techniques have disadvantages: melt extrusion requires high temperatures. Since lenalidomide has a high melting point, this may result in growing of impurities as a result of thermal decomposition. Also, the extrudates require milling in order to be useful for preparing pharmaceutical formulations thereof. Spray drying is disadvantageous, because in solvents that are suitable for spray drying, lenalidomide has low solubility. Therefore, spray drying requires copious amounts of solvents, making the process unsuitable for carrying out on a commercial scale. WO2009114601 discloses dispersions of lenalidomide in povidone. During the process to prepare these dispersions, a mixture of dimethylformamide and methanol is used. Spray drying/evaporation at high temperatures is necessary to completely remove these solvents. Using methanol and dimethylformamide is undesirable, as these solvents are toxic and are undesirable for making a pharmaceutical product.

WO2010054833 relates to non-crystalline lenalidomide in the form of a storage-stable intermediate, i.e. preferably amorphous lenalidomide together with a surface stabilizer in the form of a stable intermediate or a storage-stable intermediate, containing lenalidomide and matrix material, wherein the lenalidomide is present in the form of a solid solution (i.e. molecularly disperse). These solid solutions are prepared using melt extrusion and spray drying. These pharmaceutical techniques have disadvantages: melt extrusion requires high temperatures. Since lenalidomide has a high melting point, this may result in growing of impurities as a result of thermal decomposition. Also, the extrudates require milling in order to be useful for preparing pharmaceutical formulations thereof. Spray drying is disadvantageous, because in solvents that are suitable for spray drying, lenalidomide has low solubility. Therefore, spray drying requires copious amounts of solvents, making the process unsuitable for carrying out on a commercial scale.

EP 2875817 provides a pharmaceutical composition comprising a solid composite consisting essentially of lenalidomide and a sulfonated copolymer of styrene and divinyl benzene and processes to prepare said composition. The composition according to the invention may be used as medicament, particularly for the treatment of multiple myeloma.

Amorphous lenalidomide was disclosed in WO 2009114601.The water solubility of amorphous forms is known to be higher compared to the water solubility of crystalline forms. In view of this, it would be desirable to stabilize lenalidomide in an amorphous form.

WO 2011111053 provides anhydrous polymorphic form-I of lenalidomide and an alternate process for its preparation.

WO 2012127493 provides a novel crystalline form of lenalidomide, process for its preparation and pharmaceutical compositions comprising it. The present invention also provides a novel N-methylpyrrolidone solvate of lenalidomide and process for its preparation.

WO 2016097030 relates to a pharmaceutical composition comprising an amorphous adsorbate of lenalidomide, or a pharmaceutically acceptable salt thereof, on a porous carrier and one or more pharmaceutically acceptable excipients.

WO 2017109041 relates to a pharmaceutical composition comprising amorphous lenalidomide, or a pharmaceutically acceptable salt thereof, with a synthetic antioxidant and one or more pharmaceutically acceptable excipients. The invention further relates to the process to manufacture such a composition and the use of said composition as a medicament.

Generally, the solubility of amorphous forms is higher compared to the solubility of crystalline forms, however, due to their metastable character they require special conditions during handling and storage to avoid stability and re-crystallization problems.

Thus, in view of the prior art cited above there is still a need for compositions comprising lenalidomide that do not have the problems and disadvantages mentioned above.

The objective of the present invention was therefore to overcome the above-mentioned disadvantages and provide formulations with a good flowability and high-level content uniformity based on combining of the following approaches:
a) Prevention of API micronization, and
b) Avoiding of API solubilization during final dosage form preparation to minimize the risk of its recrystallization in any of already described polymorph.

During our laboratory experiments we have surprisingly identified that a solid formulation containing crystalline non-micronized lenalidomide form A with a mixture of disintegrant or superdisintegrant provide the same dissolution profile as a commercially available pharmaceutical form based on micronized lenalidomide form B and display dissolution behavior typical for immediate release formulations.

The formulations also comply with FDA Draft Guidance for Industry Dissolution Testing and Specification Criteria for Immediate-Release Solid Oral Dosage Forms Containing Biopharmaceutics Classification System Class 1 and 3 Drugs, which means that 85% of API is dissolved within 15 minutes in all tested media and volumes.

### BRIEF DESCRIPTION OF THE INVENTION

The present invention provides for a pharmaceutical composition comprising lenalidomide polymorphic form A, a filler, a disintegrant and/or a superdisintegrant. The present invention further relates to a processes for preparing said composition. The composition according to the invention may be used as medicament, particularly for the treatment of multiple myeloma and myelodysplastic syndromes.

The invention additionally provides for a pharmaceutical composition obtainable by a process comprising direct mixing of lenalidomide, blending and filling into polymeric hard capsules.

### BRIEF DESCRIPTION OF THE FIGURE

FIG. 1 shows the XRD pattern of Lenalidomide polymorphic form A.

### DETAILED DESCRIPTION OF THE INVENTION

The present invention provides for a pharmaceutical composition comprising lenalidomide polymorphic form A, a filler and a disintegrant and/or a superdisintegrant. In another aspect, the present invention relates to a pharmaceutical composition, characterized in that the pharmaceutical composition comprises lenalidomide polymorphic form A and a combination of filler, disintegrant and superdisintegrant. In a preferred aspect, the present invention relates to a pharmaceutical composition, characterized in that the pharmaceutical composition comprises non-micronized lenalidomide polymorphic form A and a combination of filler, disintegrant and superdisintegrant.

The present invention further relates to a processes for preparing said composition. The composition according to the present invention is obtained by direct dry mixing, i.e. direct intimate mixing of lenalidomide with a mixture of filler, and one or more disintegrant, and/or one or more superdisintegrant; and optionally one or more lubricant.

In a preferred embodiment, the lubricant is only lastly added at the end of mixing before distribution into gelatin capsules.

More specifically the invention therefore relates to a stable pharmaceutical composition comprising non-micronized lenalidomide polymorphic form A and at least one filler and one disintegrating agent, characterized in that it appears in a solid form intended for oral administration.

The term “mixture” according to the present invention refers to physical mixtures, i.e. simple admixtures of two or more excipients typically produced by short duration low-shear processing.

The term “co-processed excipient” according to the present invention refers to a definition of the International Pharmaceutical Excipient Council (IPEC) which states that a “co-processed excipient” is a combination of two or more compendial or non-compendial excipients designed to physically modify their properties in a manner not achievable by simple physical mixing and without significant chemical change.

The term “filler” according to the present invention refers to a substance with which the bulk of a pharmaceutical composition may be increased in order to provide bulk uniformity and ingredient active content uniformity of the final pharmaceutical form, a tablet or a polymeric hard capsule. Filler further allow during the manufacturing processes a proper flow of the mixture for the active ingredients which generally do not flow properly. They also allow compression to be facilitated during the making of tablets.

A filler according to the present invention is selected from sugars, advantageously sucrose, fructose, glucose, polyols, advantageously mannitol, xylitol, sorbitol, maltitol, lactitol, polysaccharides advantageously native or pre-gelatinized starch, maltodextrins, cyclodextrins, mineral compounds, advantageously dicalcium or tricalcium phosphate either dihydrates or anhydrous, cellulose derivatives, preferably microcrystalline cellulose, lactoses either monohydrates or anhydrous, as well as their mixtures, and is more advantageously selected from, lactose, mannitol, co-processed maize starch, microcrystalline cellulose and their mixtures. The filler advantageously consists in a mixture of microcrystalline cellulose and of a constituent selected from lactose monohydrate, maize starch and co-processed starch.

The pharmaceutical composition according to the present invention comprises the filler from 5o to 95% by weight, preferably from 60 to 90% by weight, more preferably from 65 to 90% by, most preferably from 78 to 90% by weight.

Lenalidomide can be characterized as class III drug (high solubility, low permeability) based on BCS (FDA, 2005, Clinical Pharmacology and Biopharmaceutics reviews, Appl. Number 21-880, page 3). In this class of compounds, the absorption rate limiting step is the permeability of the drug substance rather than the rate of dissolution of the drug formulation. Once the API liberates from pharmaceutical form to a dissolution medium it is dissolving fast, in fact it is reaching minimum 80% dissolution in 10 min. The pharmacokinetics of lenalidomide in healthy subjects are characterized by rapid oral absorption (J Clin Pharmacol 47: 1466-1475., 2007, J Bioequiv Availab 8: 214-219, 20126), time to reach maximum serum concentration (Tₘₐₓ) of 0.77-1.0 h in healthy volunteers (Cancer Chemother Pharmacol 69: 789-797., 2012)) and 1.19 h (standard deviation 0.55) in patients with acute myeloid leukemia after oral administration for 25 mg (Blood Rev 24 Supp 1: S13-S19., 2010).

To reach fast dissolution/absorption, the formulation must allow a fast release of the API into dissolution medium or body fluids. Hence, the critical parameter of lenalidomide formulation is its ability to disintegrate fast followed by complete release of the content.

The most critical excipient of the formulation seems to be the disintegrant, preferably a combination of a disintegrant with a superdisintegrant, most preferably microcrystalline cellulose and sodium croscaramellose.

The term “disintegrant” according to the present invention refers to a therapeutically inactive excipient added to oral pharmaceutical formulations (tablets and capsules) to make them disintegrate, i.e. to promote the rapid and full release of the substance within the gastrointestinal tract by breakup of tablets or capsules. Disintegration rate of the dosage form may be the rate limiting step for the absorption of the drug.

The composition according to the present invention comprises a disintegrant. The disintegrant may advantageously be selected from sodium caramellose, calcium caramellose, cellulose, starch derivatives, preferably carboxymethyl starch, pre-gelatinized starches, native starches, polyvinyl pyrrolidone derivatives, advantageously crospovidone or copovidone, soy polysaccharides, cross-linked alginic acid, gellan gum, xanthan gum, crospovidone, calcium silicate and ion exchange resins, as well as their mixtures (cf. as an example J. Pharma Sci Rev Res. 6,1:105-109, 2011). Preferably, the disintegrant is selected from crospovidone, or sodium carboxymethylcellulose, and more preferably the disintegrant is microcrystalline cellulose.

The pharmaceutical composition according to the present invention comprises the disintegrant from 50 to 95% by weight, preferably from 60 to 90% by weight, more preferably from 65 to 85% by weight.

Microcrystalline cellulose is widely used in formulations because of its excellent flow and binding properties. It is also an effective tablet disintegrant when used in a relative amount by weight of between 10-20%.

Because of the increased demands for faster dissolution requirements, there is now available, a new generation of disintegrants referred to as “superdisintegrants” in addition to the disintegrants mentioned earlier.

The term “superdisintegrant” according to the present invention refers to an inactive ingredient with improved compressivity, compatibility offering significant disintegration improvement over disintegrants like starch and microcrystalline cellulose.

Commonly used superdisintegrants are from three major groups of compounds belonging structurally to modified starches, modified cellulose and cross-linked polyvinyl pyrrolidone.

In particular, preferred superdisintegrants according to the present invention are selected from the group consisting of modified starches, modified cellulose and cross-linked polyvinyl pyrrolidone, wherein the superdisintegrant preferably is a substituted cellulose-based superdisintegrant, wherein the substituted cellulose-based superdisintegrant most preferably is sodium croscarmellose. A most preferred superdisintegrant according to the present invention is sodium croscarmellose.

A pharmaceutical composition according to the present invention comprising the superdisintegrant is characterized in that the superdisintegrant is comprised from 1 to 40% by weight, preferably from 2 to 30% by weight, more preferably from 4 to 20% by weight, most preferably from 5 to 10% by weight.

The proper choice of disintegrant, superdisintegrant or the combination thereof has an important effect on the dissolution properties of the final composition.

The most critical component of the pharmaceutical composition according to the present invention is a disintegrant, preferably a combination of a disintegrant with a superdisintegrant, preferably a combination of microcrystalline cellulose and sodium croscaramellose.

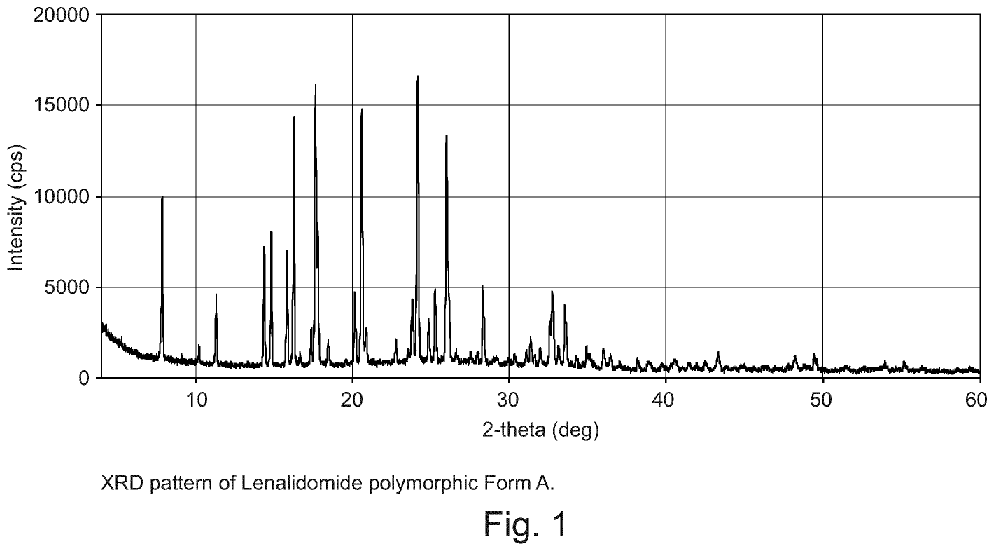

Lenalidomide form A was confirmed by XRD pattern. Identified peaks at 7.9, 14.4, 16.2, 17.6, 20.6, 24 and 26 degrees 2Θ completely correspond to form A as described in U.S. Pat. No. 746,580,0 B2 (FIG. 1).

According to a preferred embodiment according to the present invention, the composition comprises by weight: about 5 to 15% of non-micronized lenalidomide polymorphic form A, 78 to 90% of filler, advantageously split to about 1:3 of microcrystalline cellulose and pre-gelatinized maize starch or lactose monohydrate; about 5-10% of superdisintegrant, advantageously sodium croscarmellose; about 0.0 to 2.0% of lubricant, advantageously magnesium stearate or glyceryl monostearate.

Optionally, the pharmaceutical composition according to the present invention may comprise a lubricant selected from the group consisting of hydrophilic colloidal silica, magnesium stearate, glyceryl monostearate, or any combination thereof. The pharmaceutical composition according to the present invention may comprise the lubricant from 0.01 to 5.0% by weight, preferably from 0.1 to 3.0% by weight, more preferably from 0.2 to 2.0% by weight.

The components of the final composition can be added directly in form of mixtures or in a co-processed form. In case of co-processed forms, the compositions according to the present invention are preferably formed by a combination comprising lubricant, preferably glyceryl monostearate, and disintegrant, preferably microcrystalline cellulose, commercially available e.g. under the trademark Lubritose MCC® (Kerry, USA) or by a combination comprising lubricant, preferably glyceryl monostearate and disintegrant, preferably microcrystalline cellulose, and filler, preferably anhydrous lactose, commercially available e.g. under the trademark Lubritose PB®, (Kerry, USA) in combination with superdisintegrant, advantageously sodium croscarmellose.

The influence of the presence of superdisintegrant in the final composition is evident from Examples 1 to 9. When the composition contained just a disintegrant, the dissolution profile never reached more than 87% of the original product, Examples 2 to 4. When the composition further comprised a superdisintegrant, the dissolution profile rose up to 100%, Examples 5 to 7, and reached the same level as the original product, Example 8, or compositions comprising micronized lenalidomide, Example 9. The presence of superdisintegrant did not change the behavior of lenalidomide polymorph forms A and B as characterized by a similar dissolution rate. The formulation based on Form B showed about 84% of dissolution profile of formulation prepared from Form A.

Preferably the composition according to the present invention may be powder or granules. The compositions according to the present invention obtained by dry mixing were used to formulate final dosage forms by a direct fill process.

Final dosage forms according to the present invention preferably are tablets or polymeric hard capsules.

The composition according to the invention as a powder or granule may be distributed in sachets and a polymeric hard capsule, preferably selected from gelatin, hydroxypropyl methylcellulose, and pullulan.

The polymeric hard capsule may also further comprise a coloring agent, advantageously selected from pigments and oxides as well as their mixtures, more advantageously selected from titanium oxides and iron oxides, and their mixtures.

The polymeric hard capsule advantageously comprises gelatin, iron oxides and titanium dioxide.

The pharmaceutical formulations of the present invention display dissolution behavior typical for immediate release formulations with more than 80% lenalidomide API dissolved in 10 min.

The formulations also comply with FDA Draft Guidance for Industry Dissolution Testing and Specification Criteria for Immediate-Release Solid Oral Dosage Forms Containing Biopharmaceutics Classification System Class 1 and 3 Drugs, which means that 85% of API is dissolved within 15 minutes in all tested media and the volume of 500 ml.

The influence of superdisintegrant on the dissolution profiles of formulations according to the present invention will now be illustrated in a non-limiting way by the following examples.

### EXAMPLE 1

Dissolution profiles of HGCs containing 10 mg micronized API and microcrystalline cellulose

Micronized lenalidomide form A (d(0.5)=10 μm) was mixed with microcrystalline cellulose in a ratio 1:10, filled manually in Size 2 HGC and analyzed for dissolution. Dissolution experiments were run in Xtend Sotax dissolutor, in phosphate buffer pH 6.5 and 0.01 HCl pH 1.8. Sampling time intervals were 10, 15, 20, min. In house developed isocratic HPLC analytical method was used for detection of lenalidomide in dissolution media.

The results of the dissolution experiments are summarized in Table 1.

TABLE 1

| | 10 min./% | 15 min./% | 20 min./% |
|---|---|---|---|
| Dissolution buffer | dissolved API | dissolved API | dissolved API |
| 50 mM Phosphate | 99 | 101 | 101 |
| pH 6.8 | | | |
| 0.01M HCl | 99 | 100 | 101 |

The combination of micronized API and microcrystalline cellulose in a ratio 1:10 provided satisfactory dissolution profiles in both pH 1.2 and 6.8 for 10 mg strength HGCs.

### EXAMPLE 2

Dissolution profiles of HGCs containing 10 mg of non-micronized API and microcrystalline cellulose

Non-micronized lenalidomide form A (d(0.5)=30 μm) was mixed with microcrystalline cellulose in a ratio 1:10, filled manually in Size 2 HGC and analyzed for dissolution. Dissolution experiments were run in Xtend Sotax dissolutor, in phosphate buffer pH 6.5 and 0.01 HCl pH 1.8. Sampling time intervals were 10, 15, 20, min. In house developed isocratic HPLC analytical method was used for detection of lenalidomide in dissolution media.

The results of the dissolution experiments are summarized in Table 2.

TABLE 2

| | 10 min./% | 15 min./% | 20 min./% |
|---|---|---|---|
| Dissolution buffer | dissolved API | dissolved API | dissolved API |
| 50 mM Phosphate | 80 | 81 | 83 |
| pH 6.8 | | | |
| 0.01M HCl | 85 | 87 | 87 |

The combination of non-micronized API and microcrystalline cellulose in a ratio 1:10 without the use of croscarmellose provided lower dissolution profiles in both pH 1.2 and 6.8 than the formulation based on micronized API.

### EXAMPLE 3

Dissolution profiles of HGCs containing API, lactose monohydrate and microcrystalline cellulose.

20 mg of non-micronized lenalidomide form A were mixed with 20 mg microcrystalline cellulose, 60 mg lactose monohydrate and, filled manually in size 2 HGC and analyzed for dissolution as in Example 1.

The results of the dissolution experiments are summarized in Table 3.

TABLE 3

| | 10 min./% | 15 min./% | 20 min./% |
|---|---|---|---|
| Dissolution buffer | dissolved API | dissolved API | dissolved API |
| 50 mM Phosphate | 81 | 81 | 83 |
| pH 6.8 | | | |
| 0.01M HCl | 85 | 87 | 87 |

The combination of API, microcrystalline cellulose and lactose monohydrate in a ratio 1:1:3 without the use of croscarmellose provided lower dissolution profiles in both pH 1.2 and 6.8 than the formulation based on micronized API.

### EXAMPLE 4

Dissolution profiles of HGCs containing API, Starcap 1500 and microcrystalline cellulose

20 mg of non-micronized lenalidomide form A were mixed with 20 mg microcrystalline cellulose, and 60 mg Starcap 1500, filled manually in size 3 HGC and analyzed for dissolution as in Example 1.

The results of the dissolution experiments are summarized in Table 4.

TABLE 4

| | 10 min./% | 15 min./% | 20 min./% |
|---|---|---|---|
| Dissolution buffer | dissolved API | dissolved API | dissolved API |
| 50 mM Phosphate | 78 | 80 | 81 |
| pH 6.8 | | | |
| 0.01M HCl | 81 | 81 | 83 |

The combination of API, microcrystalline cellulose and Starcap 1500 in a ratio 1:1:3 without the use of croscarmellose provided lower dissolution profiles in both pH 1.2 and 6.8 than the formulation based on micronized API.

### EXAMPLE 5

Dissolution profiles of HGCs containing API, Starcap 1500, microcrystalline cellulose and croscarmellose Na.

5 mg of non-micronized lenalidomide form A were mixed with 20 mg microcrystalline cellulose, 60 mg Starcap 1500, and 10 mg croscarmellose Na, filled manually in size 3 HGC and analyzed for dissolution as in Example 1.

The results of the dissolution experiments are summarized in Table 5

TABLE 5

| | 10 min./% | 15 min./% | 20 min./% |
|---|---|---|---|
| Dissolution buffer | dissolved API | dissolved API | dissolved API |
| 50 mM Phosphate | 99 | 101 | 101 |
| pH 6.8 | | | |
| 0.01M HCl | 101 | 102 | 103 |

The combination of API, microcrystalline cellulose, Starcap 1500 and croscarmellose Na in a ratio 1:4:12:2 provided satisfactory dissolution profiles in both pH 1.2 and 6.8

### EXAMPLE 6

Dissolution profiles of HGCs containing API, Lubritose MCC, and croscarmellose Na.

5 mg of non-micronized lenalidomide form A were mixed with 80 mg Lubritose MCC and 10 mg croscarmellose Na, filled manually in size 3 HGC and analyzed for dissolution as in Example 1.

The results of the dissolution experiments are summarized in Table 6

TABLE 6

| | 10 min./% | 15 min./% | 20 min./% |
|---|---|---|---|
| Dissolution buffer | dissolved API | dissolved API | dissolved API |
| 50 mM Phosphate | 99 | 101 | 101 |
| pH 6.8 | | | |
| 0.01M HCl | 101 | 102 | 103 |

The combination of API, Lubritose MCC and croscarmellose Na in a ratio 1:16:2 provided satisfactory dissolution profiles in both pH 1.2 and 6.8.

### EXAMPLE 7

Dissolution profiles of HGCs containing API, Lubritose PB, and croscarmellose Na.

5 mg of non-micronized lenalidomide form A were mixed with 80 mg Lubritose PB and 10 mg croscarmellose Na, filled manually in size 3 HGC and analyzed for dissolution as in Example 1.

The results of the dissolution experiments are summarized in Table 7

TABLE 7

| | 10 min./% | 15 min./% | 20 min./% |
|---|---|---|---|
| Dissolution buffer | dissolved API | dissolved API | dissolved API |
| 50 mM Phosphate | 99 | 101 | 101 |
| pH 6.8 | | | |
| 0.01M HCl | 101 | 102 | 103 |

The combination of API, Lubritose PB and croscarmellose Na in a ratio 1:16:2 provided satisfactory dissolution profiles in both pH 1.2 and 6.8.

### EXAMPLE 8

Comparative dissolution profiles with Revlimid

Formulation prepared according to Example 6 was used for comparative dissolution with Revlimid HGCs in 50 mM phosphate buffer pH 6.8. The results in Table 8 show there was no difference between dissolution profile of Revlimid and tested HGC.

The results of the dissolution experiments are summarized in Table 8.

TABLE 8

| 50 mM phosphate | 10 min./% | 15 min./% | 20 min./% |
|---|---|---|---|
| buffer pH 6.8 | dissolved API | dissolved API | dissolved API |
| Revlimid | 100 | 101 | 101 |
| Tested | 99 | 100 | 101 |
| formulation | | | |

Both the reference and tested capsules provided identical comparative dissolution profile in 50 mM phosphate buffer pH 6.8.

### EXAMPLE 9

Comparative dissolution of capsules containing non-micronized lenalidomide form A and form B

Formulation prepared according to Example 6 containing either non-micronized lenalidomide form A or form B were used for comparative dissolution in 50 mM phosphate buffer pH 6.8. The results are summarized in Table 9.

TABLE 9

| 50 mM phosphate | 10 min/% | 15 min/% | 20 min/% |
|---|---|---|---|
| buffer pH 6.8 | dissolved API | dissolved API | dissolved API |
| Example 6 form A | 99 | 101 | 101 |
| Example 6 form B | 79 | 83 | 85 |

Formulations containing Form B provided about 20% lower profile than formulations containing Form A.

1. A stable pharmaceutical composition for oral administration comprising
a. from 5 to 15% by weight lenalidomide, and
b. a filler, and
c. a disintegrant, and/or
d. a superdisintegrant.

2. The stable pharmaceutical composition of claim h characterized in that lenalidomide is in crystalline form A.

3. The stable pharmaceutical composition of claim 2, characterized in that lenalidomide form A is present either in micronized or in non-micronized form.

4. The stable pharmaceutical composition of claim 3, characterized in that the filler is selected from the group consisting of sugar alcohols, starches, hydrolyzed starches, partially pregelatinized starches, or any combination thereof, and the stable pharmaceutical composition comprises the filler from 50 to 95% by weight.

5. (canceled)

6. The stable pharmaceutical composition of claim 3, characterized in that the disintegrant is selected from the group consisting of, cellulose-based excipients such as microcrystalline cellulose, low-substituted hydroxypropyl cellulose, carboxymethyl cellulose, starch derivatives, preferably carboxymethyl starch, pre-gelatinized starches, native starches, polyvinyl pyrrolidone derivatives, crospovidone, copovidone, or any combination thereof, and the stable pharmaceutical composition comprises the disintegrant from 50 to 95% by weight.

7. (canceled)

8. The stable pharmaceutical composition of claim 3, characterized in that the stable pharmaceutical composition comprises the superdisintegrant, wherein the superdisintegrant is selected from the group consisting of modified starches, modified cellulose and cross-linked polyvinyl pyrrolidone, and the stable pharmaceutical composition comprises the superdisintegrant from 1 to 40% by weight.

9. The stable pharmaceutical composition of claim 8, characterized in that the superdisintegrant is a substituted cellulose-based superdisintegrant and the substituted cellulose-based superdisintegrant is sodium croscarmellose.

10. (canceled)

11. The stable pharmaceutical composition of claim 9, wherein the disintegrant is microcrystalline cellulose and wherein the superdisintegrant is sodium croscarmellose.

12. The stable pharmaceutical composition of claim 1, characterized in that the stable pharmaceutical composition comprises additionally one or more lubricant, and the stable pharmaceutical composition comprises the one or more lubricant from 0.01 to 5.0% by weight.

13. The stable pharmaceutical composition of claim 12, characterized in that the one or more lubricant is selected from the group consisting of hydrophilic colloidal silica, magnesium stearate, glyceryl monostearate, or any combination thereof.

14. (canceled)

15. The stable pharmaceutical composition of claim 1, characterized in that the stable pharmaceutical composition comprises from 5 to 15% by weight non-micronized lenalidomide polymorphic form A, from 78 to 90% by weight filler, from 5-10% superdisintegrant, and from 0.0 to 2.0% lubricant.

16. The stable pharmaceutical composition of claim 15, wherein the filler is a mixture comprising microcrystalline cellulose and pre-gelatinized maize starch or lactose monohydrate in a ratio by weight of 1:3, and wherein the superdisintegrant is sodium croscarmellose, and wherein the lubricant is magnesium stearate or glyceryl monostearate.

17. A tablet comprising the stable pharmaceutical composition of claim 1.

18. A polymeric hard capsule comprising the stable pharmaceutical composition of claim 1 as a powder or granules, optionally, the polymeric hard capsule is made of gelatin, hydroxypropyl cellulose, pullulan, or any combination thereof.

19. (canceled)

20. A process for the preparation of a stable pharmaceutical composition of claim 1, comprising the steps of:
(a) mixing one or more filler, and one or more disintegrant, and/or one or more superdisintegrant; and optionally one or more lubricant;
(b) mixing from 5 to 15% by weight lenalidomide with the mixture obtained in step (a).

21. The process according to claim 20, wherein mixing in steps (a) and/or (b) involve short duration low-shear processing.

22. The process according to claim 20, characterized in that in step (a) a combination of lubricant and filler and/or a combination of lubricant, and/or disintegrant and filler are provided as co-processed excipients.

23. The process according to claim 22, characterized in that the combination of lubricant and disintegrant comprises glyceryl monostearate and microcrystalline cellulose.

24. The process according to claim 22, characterized in that the combination of lubricant, disintegrant and diluent comprises glyceryl monostearate, microcrystalline cellulose and anhydrous lactose.

25. A method of treating multiple myeloma or myelodysplastic syndromes, comprising administering a patient a stable pharmaceutical composition of claim 1.
